(19) United States
(12) Patent Application Publication
(10) Pub. No.: US 2016/0235880 A1
(43) Pub. Date: Aug. 18, 2016

(54) EO/PO BLOCK COPOLYMERS USEFUL AS STABILIZERS FOR PUR FOAMS

(71) Applicant: Covestro Deutschland AG, Leverkusen (DE)

(72) Inventor: Jan Schoenberger, Solingen (DE)

(21) Appl. No.: 15/137,427

(22) Filed: Apr. 25, 2016

Related U.S. Application Data
(63) Continuation of application No. 12/193,232, filed on Aug. 18, 2008.

(30) Foreign Application Priority Data
Aug. 23, 2007 (EP) 07016532.9

(51) Int. Cl. A61L 15/26; C08J 9/14; A61L 15/42; C08J 9/00

(52) U.S. Cl. CPC A61L 15/26; C08J 9/0061; C08J 9/149; A61L 15/425; C08J 2375/04; C08J 2471/02

(57) ABSTRACT

The invention relates to compositions for producing hydrophilicized polyurethane foams, in particular for wound management, wherein the composition, containing a polyurethane dispersion and specific additives, is frothed and dried.

### RELATED APPLICATIONS

This application claims benefit to European Patent Application No. 07 016 532.9, filed Aug. 23, 2007, which is incorporated herein by reference in its entirety for all useful purposes.

### BACKGROUND OF THE INVENTIONS

The invention relates to compositions for producing hydrophilicized polyurethane foams, in particular for wound management, wherein a composition containing a polyurethane dispersion and specific additives is frothed and dried.

The use of wound contact materials made of foams for treating weeping wounds is prior art. Owing to their high absorbency and their good mechanical properties, polyurethane foams produced by reaction of mixtures of diisocyanates and polyols or NCO-functional polyurethane prepolymers with water in the presence of certain catalysts and also (foam) additives are generally used. Aromatic diisocyanates are generally employed, since they are best foamable. Numerous forms of these processes are known, for example described in U.S. Pat. No. 3,978,266, U.S. Pat. No. 3,975,567 and EP-A 0 059 048. However, the aforementioned processes have the disadvantage that they require the use of reactive mixtures, containing diisocyanates or corresponding NCO-functional prepolymers, whose handling is technically inconvenient and costly, since appropriate protective measures are necessary for example.

One alternative to the above-described process, in which diisocyanates or NCO-functional polyurethane prepolymers are utilized, is a process based on polyurethane dispersions (which are essentially free of isocyanate groups) into which air is incorporated by vigorous stirring in the presence of suitable (foam) additives. So-called mechanical polyurethane foams are obtained after drying and curing. In connection with wound contact materials, such foams are described in EP-A 0 235 949 and EP-A 0 246 723, the foam either having a self-adherent polymer added to it, or being applied to a film of a self-adherent polymer. U.S. Pat. No. 4,655,210 describes the use of the aforementioned mechanical foams for wound dressings having a specific construction made up of backing, foam and skin contact layer. As described in EP-A 0 235 949, EP-A 0 246 723 and U.S. Pat. No. 4,655,210, foams were always produced from the polyurethane dispersions using additive mixtures containing ammonium stearate. This is an immense disadvantage, since in the amounts normally used ammonium stearate leads to a distinct hydrophobicization of the foams and so appreciably reduces the rate of uptake of liquid. This is unacceptable for wound contact foams in particular. In addition, ammonium stearate is thermally decomposable, and the ammonia formed has to be removed, which is technically inconvenient. On the other hand, ammonium stearate cannot simply be replaced by other stearates or completely different (foam) additives, since they fail to give a comparatively good foam structure, characterized by very fine pores in particular.

The present invention therefore has for its object to provide suitable (foam) additives which can be frothed in combination with aqueous polyurethane dispersions and, after drying, provide finely pored foams which are homogeneous even when very thick and which, compared with ammonium stearate stabilized foams, possess improved hydrophilicity and, associated therewith, a good water uptake and water vapour permeability and also are very substantially free of (thermally) detachable components such as amines.

### EMBODIMENTS OF THE INVENTION

An embodiment of the present invention is a polyurethane foam comprising an EO/PO block copolymer as stabilizer.

Another embodiment of the present invention is the above polyurethane foam, wherein said EO/PO block copolymer comprises 5% to 95% by weight of ethylene oxide units based on the sum total of all ethylene oxide and propylene oxide units and has a number-average molecular weight of 1,000 to 10,000 g/mol.

Another embodiment of the present invention is the above polyurethane foam, wherein said EO/PO block copolymer has the formula (I)

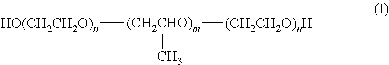

wherein
n is an integer from 2 to 160, and
m is an integer from 10 to 60.

Another embodiment of the present invention is the above polyurethane foam, wherein said polyurethane foam is hydrophilicized as well as stabilized.

Another embodiment of the present invention is the above polyurethane foam, wherein said polyurethane foam is obtained from aqueous polyurethane dispersions by physical drying.

Yet another embodiment of the present invention is a composition comprising an aqueous, anionically hydrophilicized polyurethane dispersion (I) and foam additives (II), wherein said foam additives (II) comprise an EO/PO block copolymer.

Another embodiment of the present invention is the above composition, wherein said EO/PO block copolymer comprises 5% to 95% by weight of ethylene oxide units based on the sum total of all ethylene oxide and propylene oxide units and has a number-average molecular weight of 1,000 to 10,000 g/mol.

Another embodiment of the present invention is the above composition, wherein said aqueous, anionically hydrophilicized polyurethane dispersion (I) is obtained by
A) producing isocyanate-functional prepolymers from at least
   A1) organic polyisocyanates;
   A2) polymeric polyols having number-average molecular weights in the range of from 400 to 8,000 g/mol and OH functionalities in the range of from 1.5 to 6;
   A3) optionally hydroxyl-functional compounds having molecular weights in the range of from 62 to 399 g/mol; and
   A4) optionally isocyanate-reactive, anionic, or potentially anionic and/or optionally nonionic hydrophilicizing agents;
and
B) wholly or partially reacting the free NCO groups of said isocyanate-functional prepolymers via chain extension with
   B 1) optionally with amino-functional compounds having molecular weights in the range from 32 to 400 g/mol; and/or
   B2) with isocyanate-reactive, preferably amino-functional, anionic or potentially anionic hydrophilicizing agents
   wherein said prepolymers are dispersed in water before, during or after B) and any potential ionic groups present are converted into their ionic form by partial or complete reaction with a neutralizing agent.

Another embodiment of the present invention is the above composition, wherein A1) is 1,6-hexamethylene diisocyanate, isophorone diisocyanate, the isomeric bis(4,4′-isocyanatocyclohexyl)methanes, or mixtures thereof, and A2) is a mixture of polycarbonate polyols and polytetramethylene glycol polyols, wherein the proportion of A2) which is contributed by the sum total of the polycarbonate and polytetramethylene glycol polyether polyols is at least 70% by weight.

Another embodiment of the present invention is the above composition, wherein said EO/PO block copolymers has the formula (I)

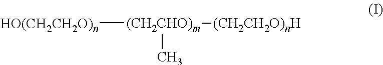

wherein
n is an integer from 2 to 160, and
m is an integer from 10 to 60.

Yet another embodiment of the present invention is a process for producing polyurethane foams, comprising frothing and drying the above composition.

Yet another embodiment of the present invention is a polyurethane foam produced by the above process.

Yet another embodiment of the present invention is wound contact material comprising the polyurethane foam.

### DESCRIPTION OF THE INVENTION

It has now been found that this object is achieved by using EO/PO block copolymers as a (foam) additive.

The present invention accordingly provides for the use of EO/PO block copolymers as stabilizers for polyurethane foams. Preferably, the use according to the present invention provides for an additional hydrophilicization of the foams as well as their stabilization. Preferably, the aforementioned polyurethane foams are of the kind obtained from aqueous polyurethane dispersions by physical drying.

The present invention further provides a process for producing polyurethane foams, wherein a composition, which likewise forms part of the subject-matter of the present invention, containing an aqueous, anionically hydrophilicized polyurethane dispersion (I) and additives (II) is frothed and dried, the foam additives (II) comprising at least an EO/PO block copolymer.

The aqueous, anionically hydrophilicized polyurethane dispersions (I) contained in the compositions which are essential to the present invention are obtainable by

A) isocyanate-functional prepolymers being produced from at least
A1) organic polyisocyanates
A2) polymeric polyols having number-average molecular weights in the range from 400 to 8000 g/mol and OH functionalities in the range from 1.5 to 6 and
A3) optionally hydroxyl-functional compounds having molecular weights in the range from 62 to 399 g/mol and
A4) optionally isocyanate-reactive, anionic or potentially anionic and/or optionally nonionic hydrophilicizing agents
   and

B) their free NCO groups then being wholly or partly reacted
B 1) optionally with amino-functional compounds having molecular weights in the range from 32 to 400 g/mol and/or
B2) with isocyanate-reactive, preferably amino-functional, anionic or potentially anionic hydrophilicizing agents
   by chain extension, and the prepolymers being dispersed in water before, during or after step B), any potentially ionic groups present being converted into the ionic form by partial or complete reaction with a neutralizing agent.

Significantly, the compounds of components A1) to A4) have no primary or secondary amino groups.

To achieve anionic hydrophilicization, A4) and/or B2) shall utilize hydrophilicizing agents that have at least one NCO-reactive group such as amino, hydroxyl or thiol groups and additionally have —COO⁻ or —SO₃⁻ or —PO₃²⁻ as anionic groups or their wholly or partly protonated acid forms as potentially anionic groups.

Preferred aqueous, anionic polyurethane dispersions (I) have a low degree of hydrophilic anionic groups, preferably from 0.1 to 15 milliequivalents per 100 g of solid resin.

To achieve good sedimentation stability, the number-average particle size of the specific polyurethane dispersions is preferably less than 750 nm and more preferably less than 550 nm, determined by laser correlation spectroscopy.

The ratio of NCO groups of compounds of component A1) to NCO-reactive groups such as amino, hydroxyl or thiol groups of compounds of components A2) to A4) is in the range from 1.05 to 3.5, preferably in the range from 1.2 to 3.0 and more preferably in the range from 1.3 to 2.5 to prepare the NCO-functional prepolymer.

The amino-functional compounds in stage B) are used in such an amount that the equivalent ratio of isocyanate-reactive amino groups of these compounds to the free isocyanate groups of the prepolymer is in the range from 40 to 150%, preferably between 50 to 125% and more preferably between 60 to 120%.

Suitable polyisocyanates for component A1) include the well-known aromatic, araliphatic, aliphatic or cycloaliphatic polyisocyanates of an NCO functionality of 2.

Examples of such suitable polyisocyanates are 1,4-butylene diisocyanate, 1,6-hexamethylene diisocyanate (HDI), isophorone diisocyanate (IPDI), 2,2,4 and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-toluylene diisocyanate, 1,5-naphthalene diisocyanate, 2,2′- and/or 2,4′- and/or 4,4′-diphenyl-methane diisocyanate, 1,3- and/or 1,4-bis(2-isocyanatoprop-2-yl)benzene (TMXDI), 1,3-bis(isocyanatomethyl)benzene (XDI), and also alkyl 2,6-diisocyanatohexanoate (lysine diisocyanates) having C₁-C₈-alkyl groups, and 4-isocyanatomethyl-1,8-octane diisocyanate (nonane triisocyanate) and triphenylmethane 4,4′,4″-triisocyanate.

As well as the aforementioned polyisocyanates, it is also possible to use, proportionally, modified diisocyanates or triisocyanates of uretdione, isocyanurate, urethane, allophanate, biuret, iminooxadiazinedione and/or oxadiazinetrione structure.

Preferably, the polyisocyanates or polyisocyanate mixtures of the aforementioned kind have exclusively aliphatically and/or cycloaliphatically attached isocyanate groups and an average NCO functionality in the range from 2 to 4, preferably in the range from 2 to 2.6 and more preferably in the range from 2 to 2.4 for the mixture.

It is particularly preferable for A1) to utilize 1,6-hexamethylene diisocyanate, isophorone diisocyanate, the isomeric bis(4,4′-isocyanatocyclohexyl)methanes, and also mixtures thereof.

A2) utilizes polymeric polyols having a number-average molecular weight Mₙ preferably in the range from 400 to 6000 g/mol and more preferably from 600 to 3000 g/mol. These preferably have an OH functionality in the range from 1.8 to 3, more preferably in the range from 1.9 to 2.1.

Such polymeric polyols are the well-known polyurethane coating technology polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols are the well-known polycondensates formed from di- and also optionally tri- and tetraols and di- and also optionally tri- and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propane-diol, 1,3-propanediol, butanediol(1,3), butanediol(1,4), hexanediol(1,6) and isomers, neopentyl glycol or neopentyl glycol hydroxypivalate, of which hexanediol(1,6) and isomers, neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl glutaric acid and/or 2,2-dimethylsuccinic acid. The corresponding anhydrides can also be used as a source of an acid.

When the average functionality of the polyol to be esterified is greater than 2, monocarboxylic acids, such as benzoic acid and hexanecarboxylic acid can be used as well in addition.

Preferred acids are aliphatic or aromatic acids of the aforementioned kind. Adipic acid, isophthalic acid and optionally trimellitic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonate diols, having number-average molecular weights Mᵣ, in the range from 400 to 8000 g/mol and preferably in the range from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxymethylcyclohexane, 2-methyl-1,3-propanediol, 2,2,4-trimethyl-1,3-pentanediol, dipropylene glycol, polypropylene glycols, dibutylene glycol, poly-butylene glycols, bisphenol A and lactone-modified diols of the aforementioned kind.

The polycarbonate diol preferably contains 40% to 100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2).

Hydroxyl-containing polycarbonates preferably have a linear construction.

A2) may likewise utilize polyether polyols. Useful polyether polyols include for example the well-known polyurethane chemistry polytetramethylene glycol polyethers as are obtainable by polymerization of tetrahydrofuran by means of cationic ring opening.

Useful polyether polyols likewise include the well-known addition products of styrene oxide, ethylene oxide, propylene oxide, butylene oxides and/or epichlorohydrin onto di- or polyfunctional starter molecules.

Useful starter molecules include all prior art compounds, for example water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

Particularly preferred embodiments of the polyurethane dispersions (I) contain as component A2) a mixture of polycarbonate polyols and polytetramethylene glycol polyols, the proportion of polycarbonate polyols in this mixture being in the range from 20% to 80% by weight and the proportion of polytetramethylene glycol polyols in this mixture being in the range from 80% to 20% by weight. Preference is given to a proportion of 30% to 75% by weight for polytetramethylene glycol polyols and to a proportion of 25% to 70% by weight for polycarbonate polyols. Particular preference is given to a proportion of 35% to 70% by weight for polytetramethylene glycol polyols and to a proportion of 30% to 65% by weight for polycarbonate polyols, each subject to the proviso that the sum total of the weight percentages for the polycarbonate and polytetramethylene glycol polyols is 100% and the proportion of component A2) which is contributed by the sum total of the polycarbonate and polytetramethylene glycol polyether polyols is at least 50% by weight, preferably 60% by weight and more preferably at least 70% by weight.

A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and also any desired mixtures thereof with one another.

Also suitable are ester diols of the specified molecular weight range such as α-hydroxy-butyl-ε-hydroxycaproic acid ester, ω-hydroxyhexyl-y-hydroxybutyric acid ester, β-hydroxyethyl adipate or bis(ε-hydroxyethyl) terephthalate.

A3) may further utilize monofunctional hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds for component A3) are 1,6-hexanediol, 1,4-butanediol, neopentyl glycol and trimethylolpropane.

An anionically or potentially anionically hydrophilicizing compound for component A4) is any compound which has at least one isocyanate-reactive group such as a hydroxyl group and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge. Useful anionically or potentially anionically hydrophilicizing compounds include mono- and dihydroxy carboxylic acids, mono- and dihydroxy sulphonic acids and also mono- and dihydroxy phosphonic acids and their salts. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃ as described in DE-A 2 446 440, page 5-9, formula I-III. Preferred anionic or potentially anionic hydrophilicizing agents for component A4) are those of the aforementioned kind that have carboxylate or carboxyl groups and/or sulphonate groups.

Particularly preferred anionic or potentially anionic hydrophilicizing agents of component A4) are those that contain carboxylate or carboxyl groups as ionic or potentially ionic groups, such as dimethylolpropionic acid, dimethylolbutyric acid and hydroxypivalic acid and salts thereof.

Useful nonionically hydrophilicizing compounds for component A4) include for example polyoxyalkylene ethers which contain at least one hydroxyl or amino group, preferably at least one hydroxyl group.

Examples are the monohydroxyl-functional polyalkylene oxide polyether alcohols containing on average 5 to 70 and preferably 7 to 55 ethylene oxide units per molecule and obtainable in a conventional manner by alkoxylation of suitable starter molecules (for example in Ullmanns Encyclopädie der technischen Chemie, 4th edition, volume 19, Verlag Chemie, Weinheim pages 31-38).

These are either pure polyethylene oxide ethers or mixed polyalkylene oxide ethers, containing at least 30 mol % and preferably at least 40 mol % of ethylene oxide units, based on all alkylene oxide units present.

Particularly preferred nonionic compounds are monofunctional mixed polyalkylene oxide polyethers having 40 to 100 mol % of ethylene oxide units and 0 to 60 mol % of propylene oxide units.

Useful starter molecules for such nonionic hydrophilicizing agents include saturated monoalcohols such as methanol, ethanol, n-propanol, isopropanol, n-butanol, isobutanol, sec-butanol, the isomers pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anis alcohol or cinnamyl alcohol, secondary monoamines such as dimethylamine, diethylamine, dipropylamine, diisopropylamine, dibutylamine, bis(2-ethylhexyl)amine, N-methylcyclohexylamine, N-ethylcyclohexylamine or dicyclohexylamine and also heterocyclic secondary amines such as morpholine, pyrrolidine, piperidine or 1H pyrazole. Preferred starter molecules are saturated monoalcohols of the aforementioned kind. Particular preference is given to using diethylene glycol monobutyl ether or n-butanol as starter molecules.

Useful alkylene oxides for the alkoxylation reaction are in particular ethylene oxide and propylene oxide, which can be used in any desired order or else in admixture in the alkoxylation reaction.

Component B1) may utilize di- or polyamines such as 1,2-ethylenediamine, 1,2-diaminopropane, 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylene-triamine, triaminononane, 1,3-xylylenediamine, 1,4-xylylenediamine, α,α,α′,α′-tetramethyl-1,3- and -1,4-xylylenediamine and 4,4-diaminodicyclohexylmethane and/or dimethylethylenediamine. It is also possible but less preferable to use hydrazine and also hydrazides such as adipohydrazide.

Component B1) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino- 1-methylam inobutane, alkanolamines such as N-aminoethylethanolamine, ethanolamine, 3-aminopropanol, neopentanolamine.

Component B1) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethyl-aminopropylamine.

Preferred compounds for component B1) are 1,2-ethylenediamine, 1,4-diaminobutane and isophoronediamine.

An anionically or potentially anionically hydrophilicizing compound for component B2) is any compound which has at least one isocyanate-reactive group, preferably an amino group, and also at least one functionality such as for example —COO⁻M⁺, —SO₃⁻M⁺, —PO(O⁻M⁺)₂ where M⁺ is for example a metal cation, Fr, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge.

Useful anionically or potentially anionically hydrophilicizing compounds are mono- and diamino carboxylic acids, mono- and diamino sulphonic acids and also mono- and diamino phosphonic acids and their salts. Examples of such anionic or potentially anionic hydrophilicizing agents are N-(2-aminoethyl)-β-alanine, 2-(2-aminoethylamino)ethanesulphonic acid, ethylenediaminepropylsulphonic acid, ethylenediaminebutylsulphonic acid, 1,2- or 1,3-propylenediamine-β-ethylsulphonic acid, glycine, alanine, taurine, lysine, 3,5-diaminobenzoic acid and the addition product of IPDA and acrylic acid (EP-A 0 916 647, Example 1). It is further possible to use cyclohexylaminopropanesulphonic acid (CAPS) from WO-A 01/88006 as anionic or potentially anionic hydrophilicizing agent.

Preferred anionic or potentially anionic hydrophilicizing agents for component B2) are those of the aforementioned kind that have carboxylate or carboxyl groups and/or sulphonate groups, such as the salts of N-(2-aminoethyl)-β-alanine, of 2-(2-aminoethylamino)ethanesulphonic acid or of the addition product of IPDA and acrylic acid (EP-A 0 916 647, Example 1).

Mixtures of anionic or potentially anionic hydrophilicizing agents and nonionic hydrophilicizing agents can also be used.

A preferred embodiment for producing the specific polyurethane dispersions utilizes components A1) to A4) and B1) to B2) in the following amounts, the individual amounts always adding up to 100% by weight:

5% to 40% by weight of component A1),

55% to 90% by weight of A2),

0.5% to 20% by weight of the sum total of components A3) and B1)

0.1% to 25% by weight of the sum total of the components A4) and B2), with 0.1% to 5% by weight of anionic or potentially anionic hydrophilicizing agents from A4) and/or B2) being used, based on the total amounts of components A1) to A4) and B1) to B2).

A particularly preferred embodiment for producing the specific polyurethane dispersions utilizes components A1) to A4) and B1) to B2) in the following amounts, the individual amounts always adding up to 100% by weight:

5% to 35% by weight of component A1),

60% to 90% by weight of A2),

0.5% to 15% by weight of the sum total of components A3) and B1)

0.1% to 15% by weight of the sum total of the components A4) and B2), with 0.2% to 4% by weight of anionic or potentially anionic hydrophilicizing agents from A4) and/or B2) being used, based on the total amounts of components A1) to A4) and B1) to B2).

A very particularly preferred embodiment for producing the specific polyurethane dispersions utilizes components A1) to A4) and B1) to B2) in the following amounts, the individual amounts always adding up to 100% by weight:

10% to 30% by weight of component A1),

65% to 85% by weight of A2),

0.5% to 14% by weight of the sum total of components A3) and B1)

0.1% to 13.5% by weight of the sum total of the components A4) and B2), with 0.5% to 3.0% by weight of anionic or potentially anionic hydrophilicizing agents from A4) and/or B2) being used, based on the total amounts of components A1) to A4) and B1) to B2).

The production of the anionically hydrophilicized polyurethane dispersions (I) can be carried out in one or more stages in homogeneous phase or, in the case of a multistage reaction, partly in disperse phase. After completely or partially conducted polyaddition from A1) to A4), a dispersing, emulsifying or dissolving step is carried out. This is followed if appropriate by a further polyaddition or modification in disperse or dissolved (homogeneous) phase.

Any prior art process can be used, examples being the prepolymer mixing process, the acetone process or the melt dispersing process. The acetone process is preferred.

Production by the acetone process typically involves the constituents A2) to A4) and the polyisocyanate component A1) being wholly or partly introduced as an initial charge to produce an isocyanate-functional polyurethane prepolymer and optionally diluted with a water-miscible but isocyanate-inert solvent and heated to temperatures in the range from 50 to 120° C. The isocyanate addition reaction can be speeded using the catalysts known in polyurethane chemistry.

Useful solvents include the customary aliphatic, keto-functional solvents such as acetone, 2-butanone, which can be added not just at the start of the production process but also later, optionally in portions. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can additionally be used or wholly or partly distilled off or in the case of N-methylpyrrolidone, N-ethylpyrrolidone remain completely in the dispersion. But preference is given to not using any other solvents apart from the customary aliphatic, keto-functional solvents.

Subsequently, any constituents of A 1) to A4) not added at the start of the reaction are added.

In the production of the polyurethane prepolymer from A1) to A4), the amount of substance ratio of isocyanate groups to with isocyanate-reactive groups is in the range from 1.05 to 3.5, preferably in the range from 1.2 to 3.0 and more preferably in the range from 1.3 to 2.5.

The reaction of components A1) to A4) to form the prepolymer is effected partially or completely, but preferably completely. Polyurethane prepolymers containing free isocyanate groups are obtained in this way, without a solvent or in solution.

The neutralizing step to effect partial or complete conversion of potentially anionic groups into anionic groups utilizes bases such as tertiary amines, for example trialkylamines having 1 to 12 and preferably 1 to 6 carbon atoms and more preferably 2 to 3 carbon atoms in every alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also bear for example hydroxyl groups, as in the case of the dialkylmonoalkanol-, alkyldialkanol- and trialkanolamines. Useful neutralizing agents further include if appropriate inorganic bases, such as aqueous ammonia solution, sodium hydroxide or potassium hydroxide.

Preference is given to ammonia, triethylamine, triethanolamine, dimethylethanolamine or diisopropylethylamine and also sodium hydroxide and potassium hydroxide, particular preference being given to sodium hydroxide and potassium hydroxide.

The bases are employed in an amount of substance which is between 50 and 125 mol % and preferably between 70 and 100 mol % of the amount of substance of the acid groups to be neutralized. Neutralization can also be effected at the same time as the dispersing step, by including the neutralizing agent in the water of dispersion.

Subsequently, in a further process step, if this has not already been done or only to some extent, the prepolymer obtained is dissolved with the aid of aliphatic ketones such as acetone or 2-butanone.

In the chain extension of stage B), NH₂— and/or NH-functional components are reacted, partially or completely, with the still remaining isocyanate groups of the prepolymer. Preferably, the chain extension/termination is carried out before dispersion in water.

Chain termination is typically carried out using amines B1) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine or suitable substituted derivatives thereof, amide-amines formed from diprimary amines and monocarboxylic acids, monoketimes of diprimary amines, primary/tertiary amines, such as N,N-dimethylaminopropylamine.

When partial or complete chain extension is carried out using anionic or potentially anionic hydrophilicizing agents conforming to definition B2) with NH₂ or NH groups, chain extension of the prepolymers is preferably carried out before dispersion.

The aminic components B1) and B2) can optionally be used in water- or solvent-diluted form in the process of the present invention, individually or in mixtures, any order of addition being possible in principle.

When water or organic solvent is used as a diluent, the diluent content of the chain-extending component used in B) is preferably in the range from 70% to 95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solution. It is preferable to add the water to the dissolved chain-extended polyurethane polymer.

The organic solvent still present in the dispersions after the dispersing step is then typically removed by distillation. Removal during the dispersing step is likewise possible.

The residual level of organic solvents in the polyurethane dispersions (I) is typically less than 1.0% by weight and preferably less than 0.5% by weight, based on the entire dispersion.

The pH of the polyurethane dispersions (I) which are essential to the present invention is typically less than 9.0, preferably less than 8.5, more preferably less than 8.0 and most preferably is in the range from 6.0 to 7.5.

The solids content of the polyurethane dispersions (I) is typically in the range from 40% to 70%, preferably in the range from 50% to 65% and more preferably in the range from 55% to 65% by weight.

The EO/PO block copolymers present in the foam additives (II) comprise the addition products—known per se in the art—of ethylene oxide and propylene oxide onto OH— or NH-functional starter molecules.

Useful starter molecules include in principle water, polyethylene glycols, polypropylene glycols, glycerol, trimethylolpropane, pentaerythritol, ethylenediamine, tolylenediamine, sorbitol, sucrose and mixtures thereof.

Starters preferably used are di- or trifunctional compounds of the aforementioned kind. Polyethylene glycol and polypropylene glycol are particularly preferred.

The respective alkylene oxide quantity and the number of EO and PO blocks can be varied to obtain block copolymers of different kinds.

In principle it is also possible that the copolymers, which basically are strictly constructed from blocks of ethylene oxide on the one hand and propylene oxide on the other, also have individual mixed blocks formed from EO and PO.

Such mixed blocks are obtained on using mixtures of EO and PO in the polyaddition reaction, so that relative to this block a random distribution of EO and PO in this block results.

The level of ethylene oxide units in the EO/PO block copolymers essential to the present invention is preferably at least 5% by weight, more preferably at least 20% by weight and most preferably at least 40% by weight based on the sum total of the ethylene oxide and propylene oxide units present in the copolymer.

The level of ethylene oxide units in the EO/PO block copolymers essential to the present invention is preferably not more than 95% by weight, more preferably not more than 90% by weight and most preferably not more than 85% by weight based on the sum total of the ethylene oxide and propylene oxide units present in the copolymer.

The number-average molecular weights of the EO/PO block copolymers essential to the present invention are preferably at least 1000 g/mol, more preferably at least 2000 g/mol and most preferably at least 5000 g/mol.

The number-average molecular weights of the EO/PO block copolymers essential to the present invention are preferably not more than 10000 g/mol, more preferably not more than 9500 g/mol and most preferably not more than 9000 g/mol.

It is particularly preferable for the EO/PO block copolymers essential to the present invention to correspond to those of the aforementioned kind and to conform to the formula (I):

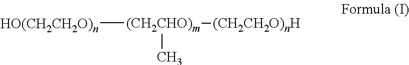

where

n is an integer from 2 to 200, preferably 60 to 180, more preferably 130 to 160, and

m is an integer from 10 to 60, preferably 25 to 45, more preferably 25 to 35.

Particular preference is given to EO/PO block copolymers of the aforementioned kind, these having an HLB value of more than 4, more preferably of more than 8 and most preferably of more than 14, the HLB being computed using the formula HLB=20·Mh/M, where Mh is the number-average molar mass of the hydrophilic moiety formed from ethylene oxide and M is the number-average molar mass of the entire molecule (Griffin, W. C.: Classification of surface active agents by HLB, J. Soc. Cosmet. Chem. 1, 1949).

The HLB value is not above 19, preferably not above 18.

As well as the EO/PO block copolymers, component (II) may contain further additives to improve foam formation, foam stability or the properties of the resulting polyurethane foam. Such further additives may in principle include any anionic, nonionic or cationic surfactant known per se. However, it is preferred that solely the EO/PO block copolymers are used as component (II).

As well as the polyurethane dispersions (I) and the foam additives (II), auxiliary and adjunct materials (III) can also be used.

Examples of such auxiliary and adjunct materials (III) are crosslinkers, thickeners or thixotroping agents, other aqueous binders, antioxidants, light stabilizers, emulsifiers, plasticizers, pigments, fillers and/or flow control agents.

Useful crosslinkers include for example unblocked polyisocyanates, amide- and amine-formaldehyde resins, phenolic resins, aldehydic and ketonic resins, examples being phenol-formaldehyde resins, resols, furan resins, urea resins, carbamic ester resins, triazine resins, melamine resins, benzoguanamine resins, cyanamide resins or aniline resins.

Commercially available thickeners can be used, such as derivatives of dextrin, of starch or of cellulose, examples being cellulose ethers or hydroxyethylcellulose, organic wholly synthetic thickeners based on polyacrylic acids, polyvinylpyrrolidones, poly(meth)acrylic compounds or polyurethanes (associative thickeners) and also inorganic thickeners, such as bentonites or silicas.

Other aqueous binders can be constructed for example of polyester, polyacrylate, polyepoxy or other polyurethane polymers. Similarly, the combination with radiation-curable binders as described for example in EP-A-0 753 531 is also possible. It is further possible to employ other anionic or nonionic dispersions, such as polyvinyl acetate, polyethylene, polystyrene, polybutadiene, polyvinyl chloride, polyacrylate and copolymer dispersions.

The compositions which are essential to the present invention typically contain, based on dry substance, 80 to 99.9 parts by weight of the polyurethane dispersion (I) and 0.1 to 20 parts by weight of the foam additive (II). Preferably, the compositions contain, based on dry substance, 85 to 99.5 parts by weight of the polyurethane dispersion (I) and 0.5 to 15 parts by weight of the foam additive (II), more preferably 90 to 99 parts by weight of dispersion (I) and 1 to 10 parts by weight of foam additive (II) and most preferably 94 to 99 parts by weight of the dispersion (I) and 1 to 6 parts by weight of foam additive (II).

The further additives added as auxiliary and adjunct materials (III) are typically used in amounts of 0 to 10 parts by weight, preferably 0.1 to 5 parts by weight and more preferably 0.1 to 1.5 parts by weight to the composition of the present invention.

The addition of the foam additives (II) and of the optional further additives to the polyurethane dispersion can take place in any desired order. The aforementioned additives may if appropriate be used as a solution or dispersion in a solvent such as water.

In principle, it is also possible to bring about a coagulation of the foam by adding coagulants as part of the auxiliary and adjunct materials. Useful coagulants include in principle all multiply cationically functional compounds.

Frothing in the process of the present invention can be accomplished by shaking or mechanical stirring of the composition or by decompressing a blowing gas.

Mechanical frothing can be effected using any desired mechanical stirring, mixing and dispersing techniques by introducing the energy necessary for frothing. Air is generally introduced, but nitrogen and other gases can also be used for this purpose.

The foam thus obtained is, in the course of frothing or thereafter, applied to a substrate or introduced into a mould and dried.

Application to a substrate can be for example by pouring or blade coating, but other conventional techniques are also possible. Multilayered application with intervening drying steps is also possible in principle.

A satisfactory drying rate for the foams is observed at a temperature as low as 20° C. However, temperatures above 30° C. are preferably used for more rapid drying and fixing of the foams. However, drying temperatures should not exceed 200° C., since undesirable yellowing of the foams can otherwise occur, inter alia. Drying in two or more stages is also possible.

Drying is generally effected using conventional heating and drying apparatus, such as (circulating air) drying cabinets, hot air or IR radiators. Drying by leading the coated substrate over heated surfaces, for example rolls, is also possible.

Application and drying can each be carried out batchwise or continuously, but an entirely continuous process is preferred.

Useful substrates include in particular papers or films which facilitate simple detachment of the foams before their use as wound contact material, for example, to cover an injured site.

Before drying, the foam densities of the polyurethane foams are typically in the range from 50 to 800 g/litre, preferably in the range from 100 to 500 g/litre and more preferably in the range from 100 to 350 g/litre (mass of all input materials [in g] based on the foam volume of one litre).

After drying, the polyurethane foams have a microporous, at least partial open-pore structure comprising intercommunicating cells. The density of the dried foams is typically below 0.4 g/cm³, preferably below 0.35 g/cm³, more preferably in the range from 0.01 to 0.3 and most preferably in the range from 0.15 to 0.3 g/cm³.

Use of the specific additives (II) provides for very rapid uptake of liquid, in particular of physiological saline. In general, 1 ml of test solution A, prepared according to DIN EN 13726-1 Part 3.2, is completely taken up in less than 25 seconds, preferably in less than 10 seconds and most preferably in less than 3 seconds.

The DIN EN 13726-1 Part 3.2 physiological saline absorbency is typically 100 and 1500%, preferably in the range from 300 to 1500% and more preferably in the range from 300 to 800% for the polyurethane foams (mass of liquid taken up, based on mass of dry foam). The DIN EN 13726-2 Part 3.2 water vapour transmission rate is typically in the range from 2000 to 8000 g/24 h*m², preferably in the range from 3000 to 8000 g/24 h*m² and more preferably in the range from 3000 to 5000 g/24 h*m².

The polyurethane foams exhibit good mechanical strength and high elasticity. Typically, strain at break is greater than 0.2 N/mm² and elongation at break is greater than 250%. Preferably, strain at break is greater than 0.4 N/mm² and the elongation at break is greater than 350% (determined according to DIN 53504).

After drying, the thickness of the polyurethane foams is typically in the range from 0.1 mm to 50 mm, preferably in the range from 0.5 mm to 20 mm, more preferably in the range from 1 mm to 10 mm and most preferably in the range from 1 mm to 5 mm.

The polyurethane foams can moreover be adhered, laminated or coated with further materials, for example materials based on hydrogels, (semi-)permeable films, coatings, hydrocolloids or other foams.

The polyurethane foams can moreover have added to them active compounds that have an effect on wound healing for example.

Owing to their advantageous properties, the polyurethane foams of the present invention are preferably used as wound contact materials or for cosmetic purposes. Wound contact materials comprising polyurethane foams within the meaning of the invention are porous materials, preferably having at least some open-cell content, which consist essentially of polyurethanes and protect wounds against microbes and environmental influences in the sense of providing a sterile covering, exhibit a rapid and high absorbence of physiological saline or wound fluid, ensure a suitable wound climate through suitable moisture permeability, and possess sufficient mechanical strength.

The present invention accordingly further provides the polyurethane foams obtainable by the process of the present invention and also for their use as a wound contact material and also in the cosmetic sector.

All the references described above are incorporated herein by in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

Unless indicated otherwise, all percentages are by weight.

Solids contents were determined in accordance with DIN-EN ISO 3251.

NCO contents were unless expressly mentioned otherwise determined volumetrically in accordance with DIN-EN ISO 11909.

Substances and Abbreviations Used:
Diaminosulphonate: NH₂—CH₂CH₂—NH—CH₂CH₂—SO₃Na (45% in water)
Desmophen® C2200: polycarbonate polyol, OH number 56 mg KOH/g, number-average molecular weight 2000 g/mol (Bayer MaterialScience AG, Leverkusen, Germany)
PolyTHF® 2000: polytetramethylene glycol polyol, OH number 56 mg KOH/g, number-average molecular weight 2000 g/mol (BASF AG, Ludwigshafen, Germany)
PolyTHF® 1000: polytetramethylene glycol polyol, OH number 112 mg KOH/g, number-average molecular weight 1000 g/mol (BASF AG, Ludwigshafen, Germany)
LB 25 polyether: monofunctional polyether based on ethylene oxide/propylene oxide, number-average molecular weight 2250 g/mol, OH number 25 mg KOH/g (Bayer MaterialScience AG, Leverkusen, Germany)
Impranil® DLU aliphatic polycarbonate-polyether-polyurethane dispersion, solids content 60%, pH 8.0 (Bayer MaterialScience AG, Leverkusen, Germany)

The determination of the average particle size (the number average is reported) of the polyurethane dispersion 1 was carried out using laser correlation spectroscopy (instrument: Malvern Zetasizer 1000, Malver Inst. Limited).

Free swell absorptive capacity was determined by absorption of physiological saline in accordance with DIN EN 13726-1 Part 3.2. The moisture vapour transition rate (MVTR) was determined in accordance with DIN EN 13726-2 Part 3.2.

The amounts reported for the foam additives are based on aqueous solutions.

### Example 1

### Preparation of Polyurethane Dispersion 1

1077.2 g of PolyTHF® 2000, 409.7 g of PolyTHF® 1000, 830.9 g of Desmophen® C2200 and 48.3 g of LB 25 polyether were heated to 70° C. in a standard stirred apparatus. Then, a mixture of 258.7 g of hexamethylene diisocyanate and 341.9 g of isophorone diisocyanate was added at 70° C. in the course of 5 min and the resulting mixture was stirred at 120° C. until the theoretical NCO value was reached or the actual NCO value had dropped slightly below the theoretical NCO value. The final prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulphonate and 1200 g of water metered in over 10 min. The mixture was subsequently stirred for 10 min. Then, a dispersion was formed by addition of 654 g of water. This was followed by removal of the solvent by distillation under reduced pressure.

The polyurethane dispersion obtained had the following properties:

Solids content: 61.6%

Particle size (LCS): 528 nm

pH (23° C.): 7.5

### Comparative Examples V1-V10

### Production of Foams from Polyurethane Dispersion 1 and Impranil® DLU

As indicated in Table 1, polyurethane dispersion 1, prepared according to Example 1, or Impranil® DLU was mixed with various (foam) additives and frothed by means of a commercially available hand stirrer (stirrer made of bent wire) to a 0.5 or 1 litre foam volume. Thereafter, the foams were drawn down on non-stick paper by means of a blade coater at a gap height of 6 mm and dried under the stated conditions.

Only with the additive combinations containing ammonium stearate, Comparative Examples V1, V2, V3 and V10, was it possible to obtain foams which were suitable for further testing. As Table 2 reveals, however, these foams exhibited an excessive hydrophobicization and hence a very low imbibition rate for physiological saline (all >60 s or >20 s). The moisture vapour transmission rate (MVTR) is comparatively low. None of the other additives (Comparative Examples V4-V8) gave any foams at all (insufficient foam-forming power on the part of the additives).

TABLE 1

| | | (Foam) additives | | | | |
|---|---|---|---|---|---|---|
| Foam | | | Amount | | Amount | |
| No. | | Type³⁾ | [g] | Type³⁾ | [g] | Curing |
| | Polyurethane | | | | | |
| | dispersion 1 | | | | | |
| | [g] | | | | | |
| V1 | 235.0¹⁾ | A | 8.5 | B | 11.3 | 60 min |
| | | | | | | 60° C., |
| | | | | | | 10 min |
| | | | | | | 120° C. |
| V2 | as for V1 | | | | | 30 min |
| | | | | | | 60° C., |
| | | | | | | 10 min |
| | | | | | | 120° C. |
| V3 | 235.0¹⁾ | A | 8.5 | C | 0.9 | 60 min |
| V4 | 117.5²⁾ | C | 0.5 | D | 2.5 | 60° C., |
| V5 | 117.5²⁾ | C | 0.5 | E | 2.5 | 10 min |
| V6 | 117.5²⁾ | C | 0.5 | F | 2.5 | 120° C. |
| V7 | 117.5²⁾ | C | 0.5 | G | 2.5 | |
| V8 | 117.5²⁾ | C | 0.5 | H | 2.5 | |
| V9 | 117.5²⁾ | C | 0.5 | I | 2.5 | |
| | Impranil ® | | | | | |
| | DLU [g] | | | | | |
| V10 | 117.5²⁾ | A | 4.2 | B | 5.6 | 10 min |
| | | | | | | 120° C. |

¹⁾foam volume 1000 ml;
²⁾foam volume 500 ml;
³⁾A: ammonium stearate (about 30%, Stokal ® STA, Bozzetto GmbH, Krefeld, DE); B: sulphosuccinamate (about 34%, Stokal ® SR, Bozzetto GmbH, Krefeld, DE); C: bis(2-ethylhexyl) sulphosuccinate, sodium salt; D: alkylaryl polyglycol ether sulphate, Na salt (Disponil ® AES 25, Cognis Deutschland GmbH & Co. KG, Düsseldorf, DE); E: modified fatty alcohol polyglycol ether (about 75%, Disponil ® AFX 2075, Cognis Deutschland GmbH & Co. KG, Düsseldorf, DE); F: fatty alcohol polyglycol ether sulphate, Na salt (Disponil ® FES 61, Cognis Deutschland GmbH & Co. KG, Düsseldorf, DE); G: fatty alcohol polyglycol ether sulphate, Na salt (Disponil ® FES 993, Cognis Deutschland GmbH & Co. KG, Düsseldorf, DE); H: C₁₃ fatty alcohol ethoxylate (about 70%, Emulan ® TO 4070, BASF AG, Ludwigshafen, DE); I: polyoxyethylene sorbitan monolaureate

TABLE 2

| | | Free swell | |
|---|---|---|---|
| | | absorptive | |
| Foam | Imbibition | capacity | MVTR |
| No. | rate¹⁾ [s] | [g/100 cm²] | [g/m²*24 h] |
| V1 | >60 ²⁾ | 33.6 | 1493 |
| V2 | >60 ³⁾ | 26.7 | n.d. |
| V3 | >60 ⁴⁾ | 31.1 | n.d. |
| V10 | >20⁴⁾ | n.d. | n.d. |

¹⁾time for complete penetration of one millilitre of test solution A prepared as in DIN EN 13726-1 Part 3.2; test on side facing the paper;
²⁾ initial measurement;
³⁾ measurement after 4 d storage;
⁴⁾ measurement after 1 d storage

### Examples S1-S4

### Production of Foams from Polyurethane Dispersion 1 and Impranil® DLU

As indicated in Table 3, polyurethane dispersion 1, prepared according to Example 1, or Impranil® DLU was mixed with various (foam) additives and frothed by means of a commercially available hand stirrer (stirrer made of bent wire) to a 0.4 litre foam volume. Thereafter, the foams were drawn down on non-stick paper by means of a blade coater at a gap height of 6 mm and dried under the stated conditions.

Fresh white foams having good mechanical properties and a fine pore structure were obtained without exception. As is discernible from Table 4, using the specific (foam) additives has appreciably enhanced the imbibition rate with regard to physiological saline (all <5 s). In addition, all the foams exhibit good free swell absorptive capacity and also a high moisture vapour transmission rate.

TABLE 3

| | Polyurethane | (Foam) additives | | | | |
|---|---|---|---|---|---|---|
| Foam | dispersion 1 | | Amount | | Amount | |
| No. | [g] | Type³⁾ | [g] | Type³⁾ | [g] | Curing |
| S1 | 120.0¹⁾ | B | 2.5 | — | — | 20 min |
| S2 | 120.0¹⁾ | B | 5.0 | — | — | 120° C. |
| S3 | 120.0¹⁾ | B | 3.8 | A | 0.3 | |
| S4 | 120.0¹⁾ | C | 5.1 | — | — | |
| | | (Foam) additives | | | | |
| | Impranil ® | | Amount | | Amount | |
| Foam No. | DLU [g] | Type³⁾ | [g] | Type | [g] | Curing |
| S5 | 120.0²⁾ | B | 4.9 | — | — | 20 min |
| | | | | | | 120° C. |

¹⁾foam volume 400 ml;
²⁾foam volume 600 ml;
³⁾A: ammonium stearate (about 30%, Stokal ® STA, Bozzetto GmbH, Krefeld, DE); B: EO/PO block copolymer (Pluronic ® PE 6800, BASF AG, Ludwigshafen, DE); C: EO/PO block copolymer (Pluronic ® PE 10500, BASF AG, Ludwigshafen, DE)

TABLE 4

| | | Free swell | |
|---|---|---|---|
| | | absorptive | |
| Foam | Imbibition | capacity | MVTR |
| No. | rate¹⁾ [s] | [g/100 cm²] | [g/m²*24 h] |
| S1 | 3 | 35.3 | 2700 |
| S2 | 3 | 44.2 | 2900 |
| S3 | 3-5 | 43.1 | 2100 |
| S4 | 3 | 43.3 | 2700 |
| S5 | 3 | 46.2 | 3400 |

¹⁾time for complete penetration of one millilitre of test solution A prepared as in DIN EN 13726-1, Part 3.2; test on side facing the paper

1-13 (canceled)

14. A polyurethane foam comprising an EO/PO block copolymer as stabilizer.

15. The polyurethane foam of claim 14, wherein said EO/PO block copolymer comprises 5% to 95% by weight of ethylene oxide units based on the sum total of all ethylene oxide and propylene oxide units and has a number-average molecular weight of 1,000 to 10,000 g/mol.

16. The polyurethane foam of claim 14, wherein said EO/PO block copolymer has the formula (I) 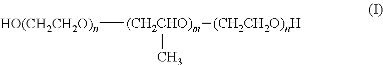
wherein
n is an integer from 2 to 160, and
m is an integer from 10 to 60.

17. The polyurethane foam of claim 1, wherein said polyurethane foam is hydrophilicized as well as stabilized.

18. The polyurethane foam of claim 14, wherein said polyurethane foam is obtained from aqueous polyurethane dispersions by physical drying.

19. A composition comprising an aqueous, anionically hydrophilicized polyurethane dispersion (I) and foam additives (II), wherein said foam additives (II) comprise an EO/PO block copolymer.

20. The composition of claim 19, wherein said EO/PO block copolymer comprises 5% to 95% by weight of ethylene oxide units based on the sum total of all ethylene oxide and propylene oxide units and has a number-average molecular weight of 1,000 to 10,000 g/mol.

21. The composition of claim 19, wherein said aqueous, anionically hydrophilicized polyurethane dispersion (I) is obtained by
A) producing isocyanate-functional prepolymers from at least
A1) organic polyisocyanates;
A2) polymeric polyols having number-average molecular weights in the range of from 400 to 8,000 g/mol and OH functionalities in the range of from 1.5 to 6;
A3) optionally hydroxyl-functional compounds having molecular weights in the range of from 62 to 399 g/mol; and
A4) optionally isocyanate-reactive, anionic, or potentially anionic and/or optionally nonionic hydrophilicizing agents;
and
B) wholly or partially reacting the free NCO groups of said isocyanate-functional prepolymers via chain extension with
B1) optionally with amino-functional compounds having molecular weights in the range from 32 to 400 g/mol; and/or
B2) with isocyanate-reactive, preferably amino-functional, anionic or potentially anionic hydrophilicizing agents
wherein said prepolymers are dispersed in water before, during or after B) and any potential ionic groups present are converted into their ionic form by partial or complete reaction with a neutralizing agent.

22. The composition of claim 21, wherein A1) is 1,6-hexamethylene diisocyanate, isophorone diisocyanate, the isomeric bis(4,4′-isocyanatocyclohexyl)methanes, or mixtures thereof, and A2) is a mixture of polycarbonate polyols and polytetramethylene glycol polyols, wherein the proportion of A2) which is contributed by the sum total of the polycarbonate and polytetramethylene glycol polyether polyols is at least 70% by weight.

23. The composition of claim 19, wherein said EO/PO block copolymers has the formula (I) 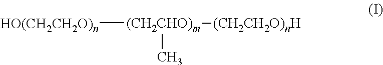
wherein
n is an integer from 2 to 160, and
m is an integer from 10 to 60.

24. A process for producing polyurethane foams, comprising frothing and drying the composition of claim 19.

25. The polyurethane foam produced by the process of claim 24.

26. A wound contact material comprising the polyurethane foam of claim 25.

27. A polyurethane foam comprising an EO/PO block copolymer as stabilizer, wherein said polyurethane foam is obtained from an aqueous, anionically hydrophilicized polyurethane dispersion (I) by mechanical frothing and physical drying,
wherein said EO/PO block copolymer comprises 40% to 85% by weight of ethylene oxide units based on the sum total of all ethylene oxide and propylene oxide units and has a number-average molecular weight of 1,000 to 10,000 g/mol,
and wherein said aqueous, anionically hydrophilicized polyurethane dispersion (I) is obtained by
A) producing isocyanate-functional prepolymers from at least
A1) organic polyisocyanates;
A2) polymeric polyols having number-average molecular weights in the range of from 400 to 8,000 g/mol and OH functionalities in the range of from 1.5 to 6;
A3) optionally hydroxyl-functional compounds having molecular weights in the range of from 62 to 399 g/mol; and
A4) optionally isocyanate-reactive, anionic, or potentially anionic and/or optionally nonionic hydrophilicizing agents;
and
B) wholly or partially reacting the free NCO groups of said isocyanate-functional prepolymers via chain extension with
B1) amino-functional compounds having molecular weights in the range from 32 to 400 g/mol; and/or
B2) with isocyanate-reactive, preferably amino-functional, anionic or potentially anionic hydrophilicizing agents,
wherein said prepolymers are dispersed in water after B) and any potential ionic groups present are converted into their ionic form by partial or complete reaction with a neutralizing agent.

28. The polyurethane foam of claim 27, wherein A1) is 1,6-hexamethylene diisocyanate, isophorone diisocyanate, the isomeric bis(4,4′-isocyanatocyclohexyl)methanes, or mixtures thereof, and A2) is a mixture of polycarbonate polyols and polytetramethylene glycol polyols, wherein the proportion of A2) which is contributed by the sum total of the polycarbonate and polytetramethylene glycol polyether polyols is at least 70% by weight.

29. The polyurethane foam of claim 27, wherein the components are present in the following amounts:
10% to 30% by weight of component A1),
65% to 85% by weight of A2),
0.5% to 14% by weight of the sum total of components A3) and B1)
0.1% to 13.5% by weight of the sum total of the components A4) and B2), with 0.5% to 3.0% by weight of anionic or potentially anionic hydrophilicizing agents from A4) and/or B2) being used, based on the total amounts of components A1) to A4) and B1) to B2).

30. The polyurethane foam of claim 27, wherein the saline absorbency is from 300-800% of mass of liquid taken up, based on mass of dry foam.
